(19) United States
(12) Patent Application Publication
(10) Pub. No.: US 2026/0225880 A1
(43) Pub. Date: Aug. 6, 2026

(54) UTILIZING SOLID-STATE CHEMICALS FOR THE DISTRIBUTED GENERATION AND STORAGE OF HYDROGEN FROM RENEWABLE ENERGY TO MANAGE POWER AND HEAT SUPPLY

(71) Applicants: Calogero Migliore, Madrid (ES); Yuguo Wang, Lexington, KY (US)

(72) Inventors: Calogero Migliore, Madrid (ES); Yuguo Wang, Lexington, KY (US)

(73) Assignees: Calogero Migliore, Madrid (ES); Yuguo Wang, Lexington, KY (US)

(21) Appl. No.: 19/043,483

(22) Filed: Feb. 2, 2025

(51) Int. Cl. C01B 3/06; C01B 35/12; C01C 1/04; C07C 29/151; C25B 1/04; C25B 15/08; F17C 11/00

(52) U.S. Cl. CPC C01B 3/065; C01B 35/121; C01C 1/04; C07C 29/151; C25B 1/04; C25B 15/08; F17C 11/005; F17C 2221/012; F17C 2265/066; F17C 2270/0105; F17C 2270/0131; F17C 2270/0168; F17C 2270/0184; F17C 2270/0189; F17C 2270/0197

(57) ABSTRACT

The invention provides a system and method for the distributed generation and storage of hydrogen utilizing solid-state chemicals. Surplus electrical energy from renewable sources, such as solar and wind, is converted into green hydrogen, stored in the form of sodium borohydride (NaBH₄). This solid-state storage medium facilitates safe and efficient hydrogen containment, overcoming limitations in transportation and storage. The hydrogen can be regenerated for use in electricity and heat generation across residential, commercial, agricultural, and industrial applications, including refueling facilities. A cycle of NaBH₄ synthesis, hydrogen extraction, and regeneration using renewable energy ensures long-term sustainability. This innovation addresses grid intermittency, enables peak shaving, and decentralizes energy systems, contributing to energy security and a low-carbon economy. The process integrates renewable energy, electrochemical synthesis, and catalytic reactions to provide a scalable solution for modern energy challenges, supporting both immediate and long-term energy needs.

### FIELD OF THE INVENTION

The present invention relates generally to a field of chemical or physical processes. More specifically, the present invention is a method, process and system of distributed generation and storage of hydrogen designed to transform surplus electrical energy generated from renewable sources, such as solar and wind, into green hydrogen that can later be reconverted into electricity and heat. It utilizes solid-state chemicals, such as sodium borohydride (NaBH₄), for storing the produced hydrogen. The stored hydrogen is then converted into electricity and/or heat, serving a diverse array of purposes in residential, commercial, farm/agricultural and industrial settings, including hydrogen refueling facilities. The storage of hydrogen can be used for managing diurnal variations and addressing intermittency in the electrical grid. It provides the capability to store hydrogen for extended periods, ranging from days to months or even entire seasons, thereby offering a flexible solution, including peak shaving.

The extra electricity generated from renewable energy sources, like solar panels, during the day can be stored as hydrogen. In times when renewable energy is unavailable, for example, due to lack of sunshine in the case of solar panels, electricity and/or heat is produced from the stored hydrogen.

### BACKGROUND

The field of chemical or physical processes is technologically important to several industries, business organizations, and/or individuals. In particular, the use of chemical or physical processes is prevalent for method and system for collecting solar power, storing the collected solar power as hydrogen, and for the storage and supply of hydrogen by using solid state chemicals.

The increasing energy demand since the industrial revolution has been mainly met by fossil fuels up to now. However, the utilization of fossil fuels has had many serious consequences such as global warming, climate change, damage to the ozone layer, acid rain, and environmental pollution. Besides the negative impact of environmental damage on people caused by fossil fuels, the finite reserves of oil and natural gas made scientists search for alternative fuels and new energy resources. The main properties of an ideal fuel include being environmentally friendly, renewable, light, having high calorific value, and capable of being produced, stored, and used in a safe, clean, and economical way. As a fuel, hydrogen meets almost all these criteria and with further research and development can be expected to fulfill the remainder. Hydrogen energy is also referred to as “independence fuel” since it eliminates the countries' dependence on the importation of oil and natural gas. Hydrogen can be used in transportation, spacecraft, electricity generation, industry, houses, and any other application where fossil fuels are currently used. However, the transportation and storage of hydrogen pose significant challenges in the hydrogen economy, hindering the development of hydrogen-related technologies for the effective deployment of the hydrogen value chain. Furthermore, in the future hydrogen economy, the bottleneck for hydrogen-related technology development is the transportation and storage of hydrogen.

Existing techniques for the storage and supply of hydrogen are deficient with regard to several aspects. For instance, current technologies do not provide a safe and efficient way of transporting hydrogen over long distances and for international import and export within the hydrogen supply chain. One of the primary challenges is the low volumetric energy density of hydrogen, which makes its storage problematic. Being the lightest and simplest element, even lighter than helium, hydrogen has a tendency to easily escape into the atmosphere, intensifying the difficulties associated with its storage.

Therefore, there is a need for improved method and system for the storage and supply of hydrogen, and distributed generation and storage of hydrogen designed to transform surplus electrical energy generated from renewable sources, such as solar and wind, into green hydrogen that can later be reconverted into electricity and/or heat using solid state chemicals is a viable solution to overcome the aforementioned problems and limitations. Furthermore, the stored hydrogen can also be used to generate electricity to meet the electrical grid's diurnal or seasonal demand swing.

Diurnal electrical demand swing refers to the daily fluctuation in electricity consumption on the power grid, which is influenced by human activities and natural patterns like day and night.

In the early morning hours, the demand for electricity is generally low. Most businesses are closed, and many people are still asleep. However, as people begin to wake up and start their day, the demand begins to increase gradually.

As businesses open and people begin their work, there's a surge in electricity consumption. Offices, factories, schools, and other commercial entities contribute significantly to this increase.

Additionally, the use of heating or cooling systems, depending on the season, also contributes to higher electrical demand.

In the late afternoon to early evening, time period often witnesses the peak of daily electricity demand. People return home from work, leading to increased use of household appliances, lighting, and HVAC systems. Also, many businesses are still operational during this period, contributing to the high demand.

In the late evening to night, and as the night progresses, businesses close, and people go to bed, leading to a decrease in electricity consumption.

The demand goes back to being low, similar to the early morning hours, and remains that way until the cycle repeats the next day.

Understanding and managing these diurnal swings and intermittency is crucial for electricity providers to ensure grid stability and reliability, as well as for economic dispatch of electricity, where the most cost-effective generation sources are used to meet the demand. In some cases, such as natural gas storage for power plants or pumped hydro storage for electricity generation, excess energy is stored during periods of low demand and released during peak times to help balance fluctuations in energy supply and demand. In this patent disclosure, extra electricity generated from renewable sources is used to produce green hydrogen, and then hydrogen is stored in the form of solid-state chemical, which is further used for diurnal or seasonal peak shaving through hydrogen fuel cell for electricity generation. Additionally, hydrogen can be used to address heating demand. This can be achieved by directly utilizing hydrogen, either alone or blended with natural gas or biogas, to meet both regular and peak heating demand on a diurnal and seasonal basis. The hydrogen source is hydrogen released from the solid state chemical through reacting with water to produce hydrogen.

The integration of solid-state chemical storage for hydrogen provides a safer, more efficient solution to the transportation and storage challenges faced by current hydrogen technologies. By converting surplus renewable energy into hydrogen and subsequently storing it in a solid-state form, this method minimizes the risk of hydrogen leakage and improves the volumetric energy density. This innovation not only enhances the practicality of hydrogen as a fuel but also supports the stability of the electrical grid by offering a flexible and responsive means of energy storage and supply. The utilization of solid-state chemicals thus represents a significant advancement in the hydrogen economy, promoting the broader adoption of hydrogen as a key energy carrier in a sustainable future.

Furthermore, the deployment of this technology can facilitate the decentralization of energy systems. Distributed generation and storage of hydrogen using solid-state chemicals enable localized energy production and consumption, reducing the reliance on centralized power plants and extensive grid infrastructure. This decentralization can lead to greater energy security, as communities become more self-sufficient in their energy needs, and also mitigate the impact of power outages. Moreover, this approach aligns with the global shift towards renewable energy sources, contributing to the reduction of greenhouse gas emissions and fostering the transition to a low-carbon economy. By addressing both the storage and transportation hurdles, this innovative method can play a crucial role in realizing a sustainable and resilient energy future.

### SUMMARY

This summary is provided to introduce a selection of concepts in a simplified form that are further described below in the Detailed Description of the Invention. This summary is not intended to identify key or essential features of the claimed subject matter. Nor is this summary intended to be used to limit the claimed subject matter's scope.

This invention discloses a novel method and process for distributed hydrogen generation and storage by utilizing excess electrical energy generated from renewable sources, such as solar and wind, to produce a safe solid hydrogen storage medium as NaBH₄, which can be subsequently utilized to produce hydrogen gas for meeting the electricity needs such as for diurnal or seasonal peak shaving, and heat requirements of a diverse array of applications, including residential, commercial, farm/agricultural, and industrial settings, including hydrogen refueling facilities.

According to some aspects, a method for storage and supply of hydrogen using solid state chemicals is disclosed. Further, the method may include a step of synthesizing a sodium borohydride (NaBH₄) mass from a natural resource or from sodium metaborate (NaBO₂) and water through an electrochemical method. Further, the method may include a step of producing at least one solid state hydrogen carrier using the sodium borohydride mass. Further, the method may include a step of chemically extracting a hydrogen volume from the at least one solid state hydrogen carrier by reacting the sodium borohydride mass with water in a presence of a catalyst. Further, the chemically extracting may produce a mass of sodium metaborate as a byproduct. Further, the method may include a step to regenerate the sodium borohydride mass from the sodium metaborate mass using electrochemical means. Further, the method may include a step of processing the hydrogen volume in a dehydrator to remove moisture to obtain a usable quantity of hydrogen. Further, the method may include a step to store the usable quantity of hydrogen in a hydrogen storage unit.

The proposed distributed energy generation system involves the utilization of multiple sources of energy, particularly photovoltaic (PV) panels and wind, to generate electricity for self-consumption. The system can be adapted to any type of renewable energy or combination thereof. Any excess electricity produced is harnessed to produce sodium borohydride (NaBH₄) from sodium metaborate and water through electrochemical method as shown below.

The aqueous electrosynthesis of NaBH₄, starting from NaBO₂, is described by the following reaction occurring at the cathode and anode in an electrochemical cell, where E° stands for the standard electrode potential.

### Cathode Reaction:

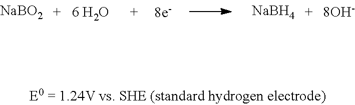

### Anode Reaction:

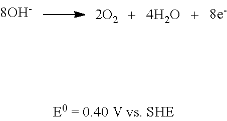

Sodium borohydride, a solid-state metal borohydride, is a solid hydrogen storage medium that contains 10.6 wt % of hydrogen. This solid storage medium offers the advantage of being non-flammable at ambient temperature and pressure, making it safer in comparison to compressed hydrogen.

In instances where the primary energy source is not available due to intermittency or any other reason, for instance absence of sunlight in the case of photovoltaic panels, or unfavorable wind conditions for wind power, the stored hydrogen fulfills the dual roles of generating electricity and heat. The synthesized sodium borohydride (NaBH₄) is combined with water (H₂O) as a co-reactant to produce hydrogen (H₂) gas in the presence of a catalyst. This reaction yields hydrogen gas equivalent to storing hydrogen in the solid state NaBH₄ with hydrogen content of 21.3 wt %. The generated hydrogen is subsequently utilized on-site or can serve as a direct heat source and/or be utilized in a fuel cell to generate electricity, which is used as needed within the premises.

The reaction between sodium borohydride and water proceeds as follows:

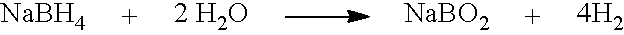

Sodium metaborate (NaBO₂) is produced as a byproduct, which can be regenerated into sodium borohydride through electrochemical means and using water, with the ideal energy source for this regeneration being renewable energy, for instance, from the solar panels in the case of PV, or from wind, or hydroelectricity, or geothermal.

The produced hydrogen then undergoes compression and further processing in a dehydrator to eliminate any remaining moisture. This purified hydrogen can be utilized in a hydrogen fuel cell to generate electricity for the electrical grid peak shaving or for residential use, or employed directly, or blended with natural gas or biogas, for heating purposes. Part of the electricity generated in the fuel cell can be also used for the electrochemical regeneration of sodium borohydride from sodium metaborate (NaBO₂) as described before.

All the water needed for each stage of the process can be obtained from an external source or, when possible, collected on the premises, such as by extracting it from an underground reservoir/well.

As an alternative configuration for the distributed hydrogen generation and storage process, the sodium borohydride (NaBH₄) can be externally synthesized and then transported to the site location for one-time deployment. Hydrogen is generated through the reaction of sodium borohydride with water as a co-reactant, which is then utilized on-site as a direct heat source and/or as a fuel source for a fuel cell, meeting energy needs when the primary energy supply is unavailable.

According to some aspects, a method for collecting renewable energy such as solar energy through photovoltaic panel is used for synthesizing a sodium borohydride (NaBH₄) mass from sodium metaborate (NaBO₂) and water. Further, the method may include a step of chemically extracting a hydrogen volume by reacting the sodium borohydride mass with water in the presence of a catalyst. Further, the chemically extracting of hydrogen may produce a mass of sodium metaborate as a byproduct. Further, the method may include a step to regenerate the sodium borohydride mass from the sodium metaborate mass using electrochemical means. Further, the method may include steps of compression and processing the hydrogen volume in a dehydrator to remove moisture to obtain a usable quantity of hydrogen. Further, the method may include a step to feed the hydrogen into a fuel cell to generate electricity for local use. Further, the electricity produced by the hydrogen fuel cell can also be used to supply electrical power to the grid during peak demand periods. Further, hydrogen can also be used to charge into the gas grid. Further, hydrogen can be burned locally for heating purposes. Further, the hydrogen can be used to refuel vehicles through the utilization of hydrogen refueling facilities. Further, the hydrogen can be combined with nitrogen to produce ammonia. Further, the hydrogen can be combined with carbon dioxide (CO₂) to produce methanol. Further, the hydrogen can be used together with carbon dioxide (CO₂) to produce synthetic fuels and chemicals. Further, the hydrogen can be combined with carbon monoxide (CO), which was initially generated from carbon dioxide (CO₂), to make synthesis gas. This synthesis gas can be utilized in processes such as those employing the Fischer-Tropsch reaction to produce synthetic fuels, wax, and chemicals.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF DRAWINGS

The accompanying drawings, which are incorporated in and constitute a part of this disclosure, illustrate various embodiments of the present invention.

Furthermore, the drawings may contain text or captions that may explain certain embodiments of the present disclosure. This text is included for illustrative, non-limiting, explanatory purposes of certain embodiments detailed in the present disclosure.

FIG. 1 illustrates a flowchart of a method for storage and supply of hydrogen using solid state chemicals according to one or more embodiments herein.

FIG. 2 illustrates a flow diagram of a system for storage and supply of hydrogen using solid state chemicals according to one or more embodiments herein.

FIG. 3 shows a flowchart of a novel method for distributed generation and storage of green hydrogen. This flowchart outlines a schematic for a green hydrogen solution that combines heat and power for use in residential, commercial, farm and industrial applications using solid state chemicals, in accordance with some embodiments. Green hydrogen is produced from water and renewable energy sources and stored as sodium borohydride (NaBH₄), which can be synthesized from readily available natural resource, borax. Subsequently, this stored hydrogen is converted into electricity and heat, catering to a wide range of purposes in residential, commercial, farm/agricultural and industrial settings, including hydrogen refueling facilities. Seawater can also serve as a resource for synthesizing sodium borohydride, especially in offshore applications in combination with desalination processes using renewable energy.

FIG. 4 shows a schematic of a decentralized system for generating and storing green hydrogen, integrating both heat and power components for residential applications. However, its versatility extends to a wide array of applications, encompassing commercial, agricultural, and industrial settings, such as hydrogen refueling stations. During the daytime, solar panels provide electricity, with the surplus power used to synthesize sodium borohydride from sodium metaborate and water through an electrochemical process. This process enables hydrogen to be stored in the form of sodium borohydride for later use. The primary energy needs are met by the solar panels, and only excess energy is used to synthesize sodium borohydride (NaBH₄) from sodium metaborate (NaBO₂) and water. Sodium metaborate and water are readily available resources that can be placed on-site next to the energy utilization facility. Sodium borohydride serves as a safe solid medium for hydrogen storage. The water required for every step of the process can be sourced from an external supply or, when feasible, produced on-site, for example, by extracting it from an underground reservoir/well.

In instances of intermittent or absent sunlight, the stored hydrogen fulfills the dual roles of generating electricity and heat. When sunshine is unavailable, hydrogen is generated through the catalytic hydrolysis of sodium borohydride using water. This process involves the reaction of sodium borohydride with water in the presence of a catalyst, resulting in the production of H₂ at the point of use. Additionally, sodium metaborate (NaBO₂) is produced as a byproduct, which can be used for synthesis of sodium borohydride through electrochemical means and using water, with the ideal energy source for this regeneration being renewable energy, such as surplus power from the solar panels in the case of photovoltaic (PV), or from wind, or hydroelectricity, or geothermal.

The produced hydrogen then undergoes compression and further processing in a dehydrator to eliminate any remaining moisture. This purified hydrogen can be utilized for heating purposes within the residential setting. Furthermore, the purified hydrogen can also be directly fed into a hydrogen fuel cell to generate electricity for the residential setting or nearby premises

In situations where excess renewable energy is unavailable to power the electrochemical regeneration of NaBH₄ from sodium metaborate using water, the power generated by the fuel cell can also be utilized as required for the electrochemical regeneration process. This approach allows for hydrogen storage that can extend from days to months or even entire seasons, making it a versatile solution.

In summary, this system represents a decentralized approach to generating and storing green hydrogen, integrating both heat and power supply for use in residential, commercial, farm/agricultural and industrial applications using solid state chemicals, while remaining independent of the external gas and power grid infrastructure. It effectively utilizes surplus renewable energy and available water resources to generate and store hydrogen in the form of sodium borohydride.

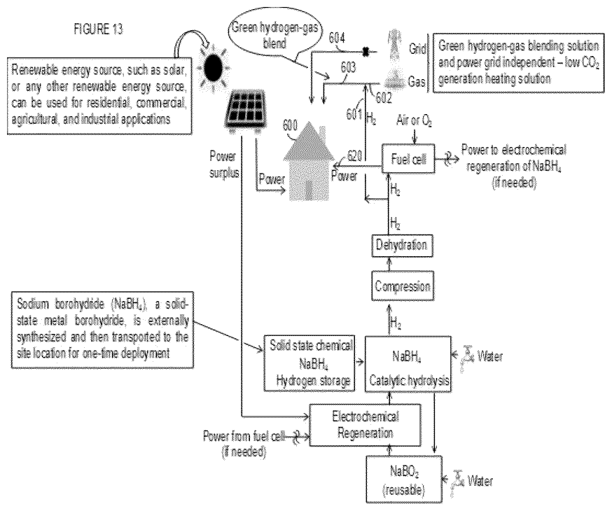

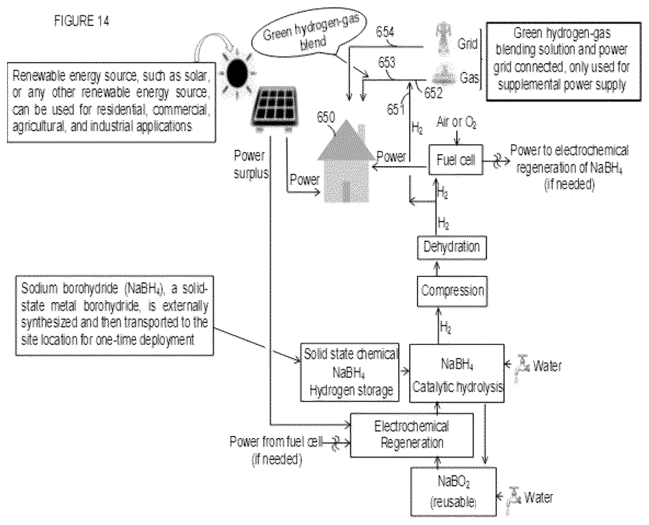

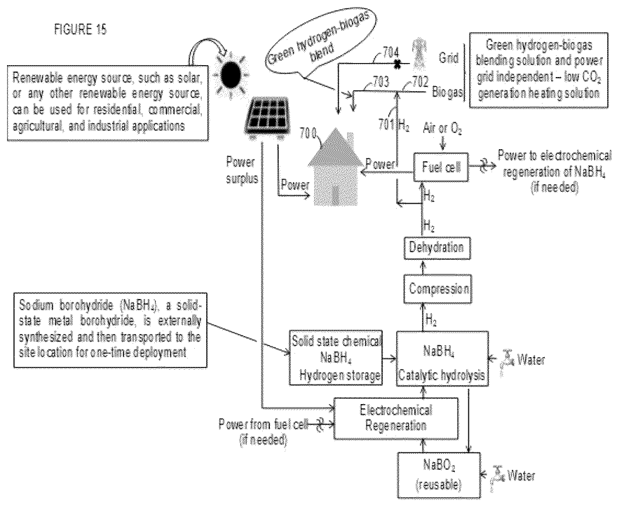

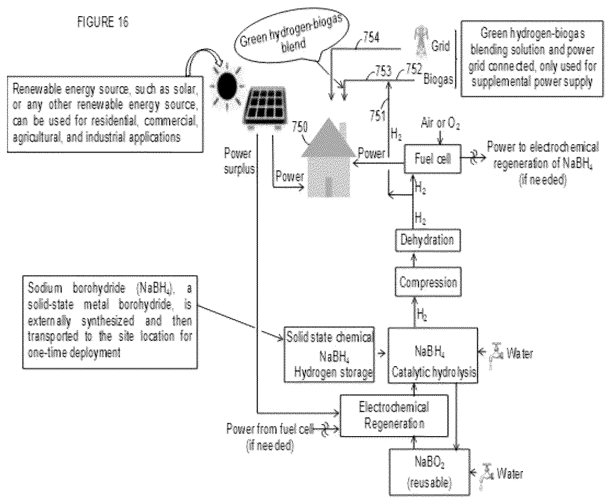

FIG. 5 shows a schematic of a decentralized system for generating and storing green hydrogen, integrating both heat and power components for residential, farm or industrial applications. This configuration aligns with the description outlined in FIG. 4, with the distinction that the external gas and power grid connections are primarily utilized as supplementary sources for power and heat. This system utilizes distinct heating sources for hydrogen and gas.

FIG. 6 shows a schematic of a decentralized system for generating and storing green hydrogen, integrating both heat and power components for residential, farm or industrial applications. This configuration aligns with the description outlined in FIG. 4, with the distinction that the green hydrogen is blended with externally supplied natural gas using the green hydrogen-gas mixture for heating purposes keeping the system independent of the external power grid infrastructure. This system is a low CO₂ generation heating solution.

FIG. 7 shows a schematic of a decentralized system for generating and storing green hydrogen, integrating both heat and power components for residential, farm or industrial applications. This configuration aligns with the description outlined in FIG. 4, with the distinction that the green hydrogen is blended with externally supplied natural gas using the green hydrogen-gas mixture for heating purposes, and connected to the external power grid primarily utilized as supplementary source for power.

FIG. 8 shows a schematic of a decentralized system for generating and storing green hydrogen, integrating both heat and power components for residential, farm or industrial applications. This configuration aligns with the description outlined in FIG. 4, with the distinction that the green hydrogen is blended with on-site produced or externally supplied biogas using the green hydrogen-biogas mixture for heating purposes keeping the system independent of the external power grid infrastructure. This system is a low CO₂ generation heating solution.

FIG. 9 shows a schematic of a decentralized system for generating and storing green hydrogen, integrating both heat and power components for residential, farm or industrial applications. This configuration aligns with the description outlined in FIG. 4, with the distinction that the green hydrogen is blended with on-site produced or externally supplied biogas using the green hydrogen-biogas mixture for heating purposes, and connected to the external power grid primarily utilized as supplementary source for power.

FIG. 10 shows a flowchart of a novel method for distributed generation and storage of green hydrogen. This flowchart outlines a schematic for a green hydrogen solution that combines heat and power for use in residential, commercial, farm/agricultural and industrial applications using solid state chemicals, in accordance with some embodiments. Green hydrogen is produced from water and renewable energy sources. The solution utilizes solid-state chemicals, such as sodium borohydride (NaBH₄). Sodium borohydride (NaBH₄), a solid-state metal borohydride, is externally synthesized and then transported to the site location for one-time deployment. When the renewable energy source, such as sunlight, is not available, for example, due to intermittency, hydrogen is generated through the catalytic hydrolysis of sodium borohydride using water. This process involves the reaction of sodium borohydride with water in the presence of a catalyst, resulting in the production of H₂ at the point of use. Additionally, sodium metaborate (NaBO₂) is produced as a byproduct, which can be used for synthesis of sodium borohydride through electrochemical means and using water, with the ideal energy source for this regeneration being renewable energy, such as surplus power from the solar panels in the case of photovoltaic (PV), or from wind, or hydroelectricity, or geothermal. Subsequently, the produced hydrogen is efficiently converted into electricity and heat, catering to a wide range of purposes in residential, commercial, farm/agricultural and industrial settings, including hydrogen refueling facilities. Although the description is based on sodium borohydride, this solution can also use any other solid-state metal borohydrides and same as NaBH₄ would be externally synthesized and then transported to the site location for one-time deployment.

FIG. 11 shows a schematic of a decentralized system for generating and storing green hydrogen, integrating both heat and power components for residential applications. However, its versatility extends to a wide array of applications, encompassing commercial, agricultural, and industrial settings, such as hydrogen refueling stations. During the daytime, solar panels provide electricity, with surplus power stored as hydrogen for later use. Sodium borohydride (NaBH₄), a solid-state metal borohydride, is externally synthesized and then transported to the site location for one-time deployment. NaBH₄ is a solid substance that can be placed on-site next to the energy utilization facility. The water required for every step of the process can be sourced from an external supply or, when feasible, produced on-site, for example, by extracting it from an underground reservoir/well.

In instances of intermittent or absent sunlight, the stored hydrogen fulfills the dual roles of generating electricity and heat. When sunshine is unavailable, hydrogen is generated through the catalytic hydrolysis of sodium borohydride using water. This process involves the reaction of sodium borohydride with water in the presence of a catalyst, resulting in the production of H₂ at the point of use. Additionally, sodium metaborate (NaBO₂) is produced as a byproduct, which can be used for synthesis of sodium borohydride through electrochemical means, with the energy source for this regeneration coming from the surplus power from the solar panels. The primary energy needs are met by the solar panels, and only excess energy is used to electrochemically regenerate sodium borohydride from sodium metaborate and water.

The produced hydrogen then undergoes compression and further processing in a dehydrator to eliminate any remaining moisture. This purified hydrogen can be utilized for heating purposes within the residential setting. Furthermore, the purified hydrogen can be directly fed into a hydrogen fuel cell to generate electricity for the residential setting. This approach allows for hydrogen storage that can extend from days to months or even entire seasons, making it a versatile solution.

In summary, this system represents a decentralized approach to generating and storing green hydrogen, integrating heat and power supply for residential use, while remaining largely independent of the external gas and power grid infrastructure. It effectively utilizes surplus renewable energy and available water resources to generate and store hydrogen in the form of sodium borohydride.

FIG. 12 shows a schematic of a decentralized system for generating and storing green hydrogen, integrating both heat and power components for residential, farm or industrial applications. This configuration aligns with the description outlined in FIG. 11, with the distinction that the external gas and power grid connections are primarily utilized as supplementary sources for power and heat. This system utilizes distinct heating sources for hydrogen and gas.

FIG. 13 shows a schematic of a decentralized system for generating and storing green hydrogen, integrating both heat and power components for residential, farm or industrial applications. This configuration aligns with the description outlined in FIG. 11, with the distinction that the green hydrogen is blended with externally supplied natural gas using the green hydrogen-gas mixture for heating purposes keeping the system independent of the external power grid infrastructure. This system is a low CO₂ generation heating solution.

FIG. 14 shows a schematic of a decentralized system for generating and storing green hydrogen, integrating both heat and power components for residential, farm or industrial applications. This configuration aligns with the description outlined in FIG. 11, with the distinction that the green hydrogen is blended with externally supplied natural gas using the green hydrogen-gas mixture for heating purposes, and connected to the external power grid primarily utilized as supplementary source for power.

FIG. 15 shows a schematic of a decentralized system for generating and storing green hydrogen, integrating both heat and power components for residential, farm or industrial applications. This configuration aligns with the description outlined in FIG. 11, with the distinction that the green hydrogen is blended with on-site produced or externally supplied biogas using the green hydrogen-biogas mixture for heating purposes keeping the system independent of the external power grid infrastructure. This system is a low CO₂ generation heating solution.

FIG. 16 shows a schematic of a decentralized system for generating and storing green hydrogen, integrating both heat and power components for residential, farm or industrial applications. This configuration aligns with the description outlined in FIG. 11, with the distinction that the green hydrogen is blended with on-site produced or externally supplied biogas using the green hydrogen-biogas mixture for heating purposes, and connected to the external power grid primarily utilized as supplementary source for power.

### DETAILED DESCRIPTION OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denotes “at least one”, but does not exclude a plurality unless the contextual use dictates otherwise. When used herein to join a list of items, “or” denotes “at least one of the items”, but does not exclude a plurality of items of the list. Finally, when used herein to join a list of items, “and” denotes “all of the items of the list”.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods and systems for storage and supply of hydrogen using solid state chemicals, embodiments of the present disclosure are not limited to use only in this context.

One or more embodiments herein generally relates to a method for storing, and supplying of hydrogen gas as a renewable fuel source to promote a hydrogen economy.

The present disclosure describes methods and systems that utilize excess renewable energy to produce a solid chemical that effectively stores green hydrogen for future utilization, whether for short-term or long-term purposes, spanning hours to entire seasons. Further, the disclosed systems may be configured for hydrogen storage using solid state chemicals such as sodium borohydride (NaBH₄), and converting into electricity and heat to serve a wide range of purposes in residential, commercial, farm/agricultural and industrial settings, including hydrogen refueling facilities.

As a solid hydrogen storage medium, sodium borohydride has 10.6 wt % of hydrogen, it has the advantage of being non-flammable at ambient temperature and pressure. This makes it a safe and convenient option for transporting hydrogen in solid form. When the transported medium (NaBH₄) is used for hydrogen production at its destination site, together with its co-reactant H₂O, the H₂ released is equivalent to transporting NaBH₄ in the solid state with a 21.3 wt % of hydrogen.

In comparison to the conventional approach of producing H₂ from fossil fuels, this method/process eliminates CO₂ emissions during H₂ production and storage.

The disclosed method/process also makes use of naturally occurring resources such as borax to make green hydrogen using renewable energy.

Further, the method includes synthesizing sodium borohydride (NaBH₄) from natural resources which are abundant, namely borax, seawater and silica (SiO₂). The energy consumed in the synthesis of the sodium borohydride may be from any source and particularly from a renewable source.

Further, the method includes using the synthesized sodium borohydride as a safe solid medium for storing hydrogen.

Further, the method includes reacting sodium borohydride with water in the presence of a catalyst to yield H₂ at the consumption site. The reaction also produces sodium metaborate as a byproduct, which can be regenerated back into sodium borohydride using electrochemical means. It is preferable to use energy from a renewable source to facilitate this regeneration process. Eventually, the regeneration of sodium metaborate will reach equilibrium with the needed amount of sodium borohydride, at which no further production of sodium borohydride from borax is needed. Further, the hydrogen produced from the reaction between sodium borohydride and water will undergo further processing in a dehydrator to remove any remaining moisture. Further, the purified hydrogen may be directly used onsite or may be stored using compression, liquefaction, adsorption onto a solid porous adsorbent, or any other appropriate method, in corresponding tanks. These tanks can also be transported locally to where hydrogen is used. Further, in some embodiments, the purified hydrogen can also be directly fed to a hydrogen fuel cell to generate electricity as a power source for mobile devices such as buses, cars, boats, and manned and unmanned flying vehicles. In addition to its use as a fuel source for hydrogen fuel cells, purified hydrogen can also be employed for stationary power generation, including emergency and backup power services. This can be achieved by utilizing hydrogen as a fuel in power plants, either through direct use or blending it into the fuel stream. Further, in some embodiments, hydrogen can be supplied as fuel for distributed power generators in smart grid construction. The reactants for the hydrogen fuel cell are air or oxygen and hydrogen, and the only product is water. Further, the regeneration of sodium metaborate to sodium borohydride involves adding hydrogen back into sodium metaborate. The hydrogen is preferred from green electrolysis of water, but it can also come from any other sources. Beyond this point, there is no additional production of sodium borohydride from borax needed.

Further, sodium borohydride (NaBH₄) is synthesized from natural resources which are abundant, namely borax, seawater, and silica. The energy consumed in the synthesis of the sodium borohydride is from any source and particularly from a renewable source.

Further, the synthesis of NaBH₄ may go through the commercially available Bayer process for the synthesis of sodium borohydride:

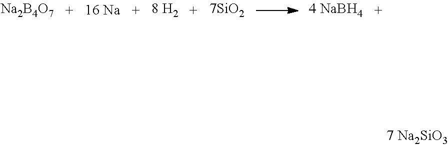

Further, the Bayer process is a one-pot synthesis that involves the combination of borax, metallic sodium, and hydrogen in the presence of silica to produce sodium borohydride.

Among chemical hydrides, sodium borohydride (NaBH₄) is a well-known hydrogen source due to its high hydrogen storage capacity of 10.6%. In combination with water, it releases even more hydrogen, making it comparable to a hydrogen carrier medium that can store up to 21.3 wt % of hydrogen:

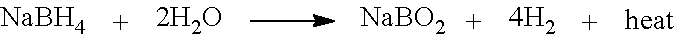

In addition to its hydrogen storage capabilities, NaBH₄ can also be safely and stably dissolved in an alkaline aqueous solution over long periods of time. When necessary, the hydrogen can be released on demand by hydrolyzing NaBH₄ using a heterogeneous catalyst. Cobalt, ruthenium, and nickel are among the catalysts that have been extensively researched. Catalysts based on non-noble metals, especially metal borides (Co—B, Ni—B, etc.) received considerable attention due to their high efficiency, low cost, and simple preparation methods.

The aqueous electrosynthesis of NaBH₄, starting from NaBO₂, is described by the following reaction occurring at the cathode and anode in an electrochemical cell.

### Cathode Reaction:

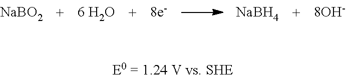

### Anode Reaction:

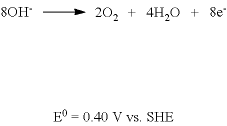

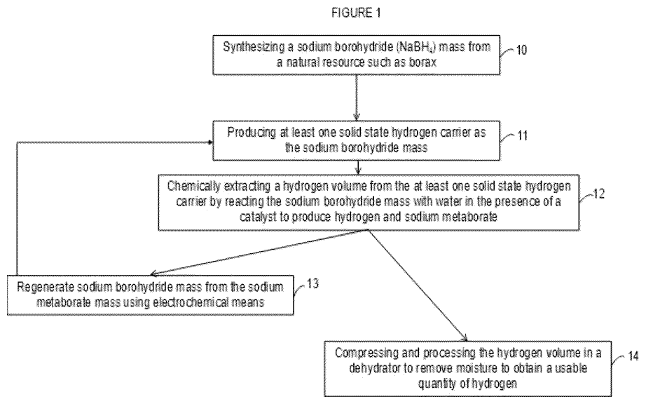

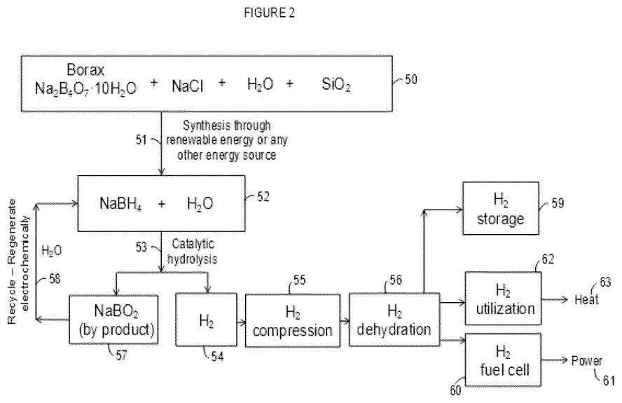

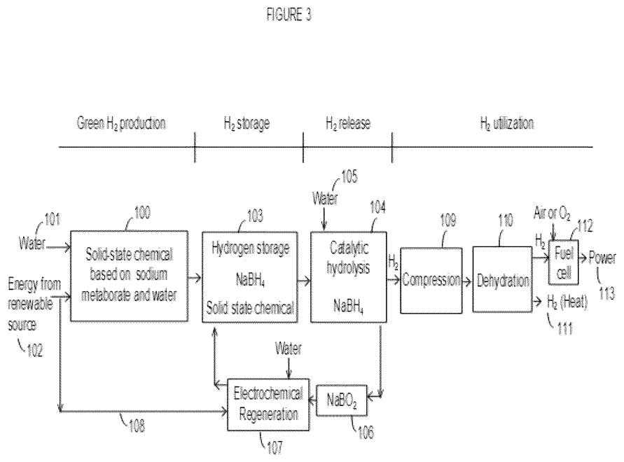

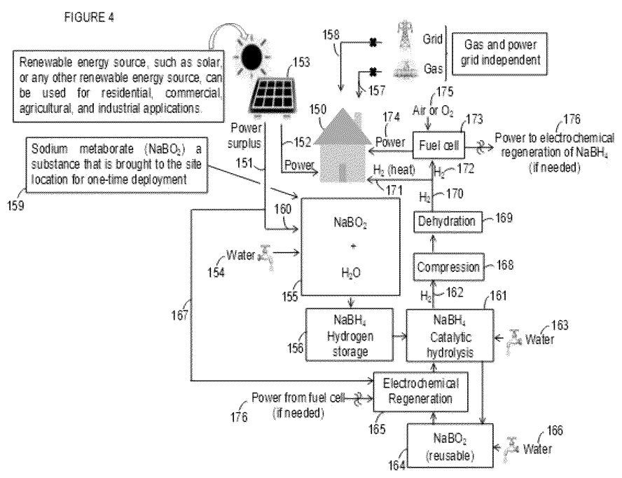

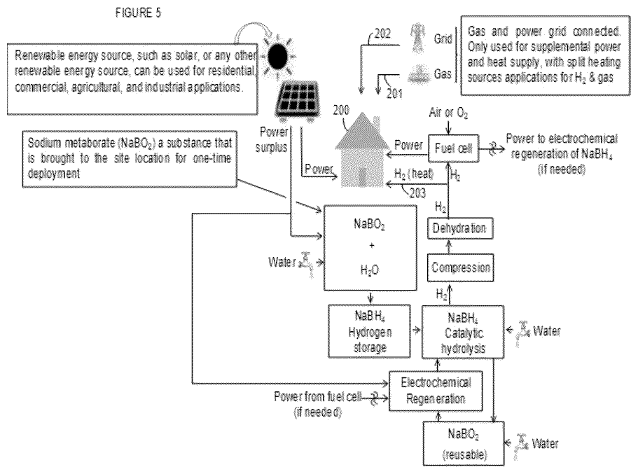

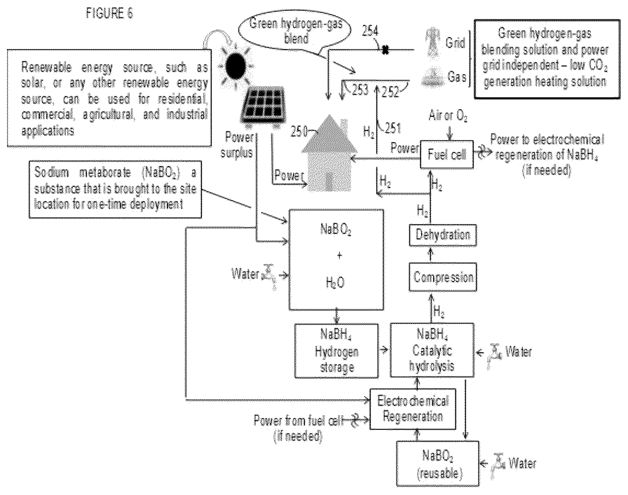

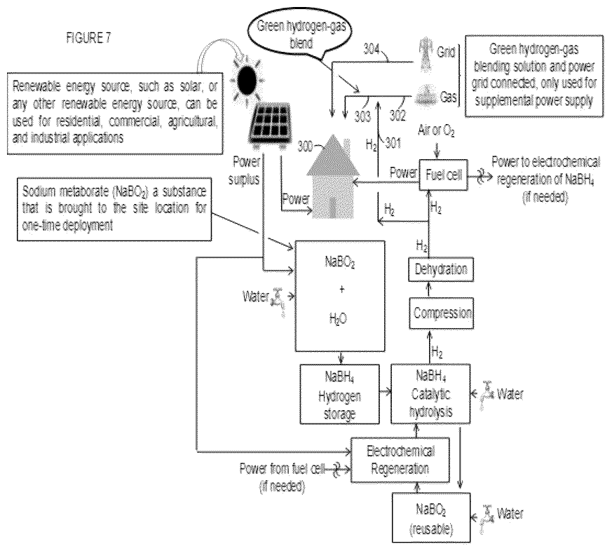

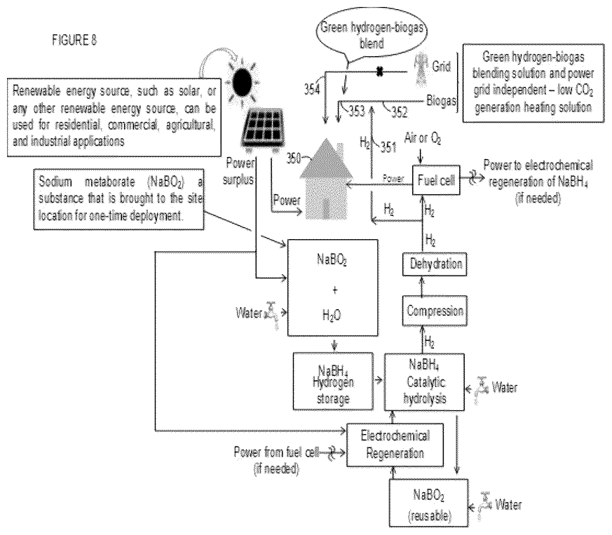

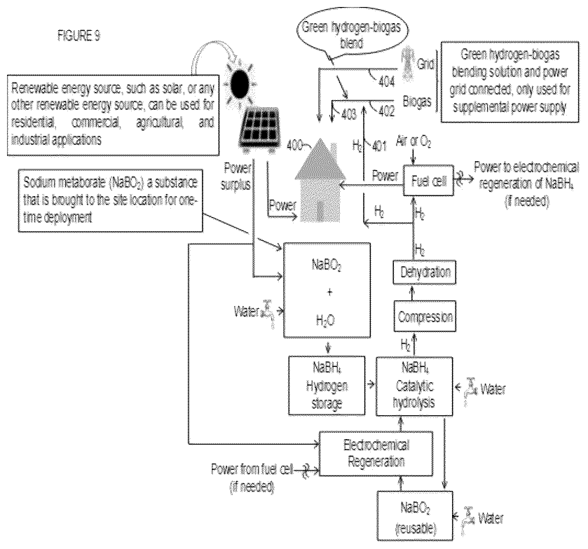

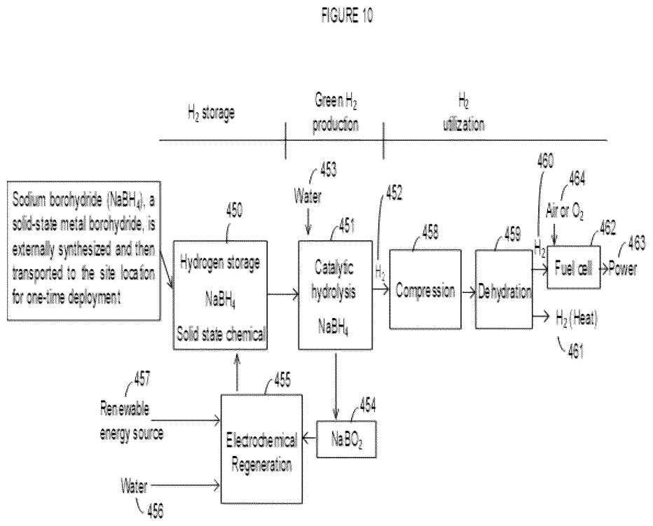

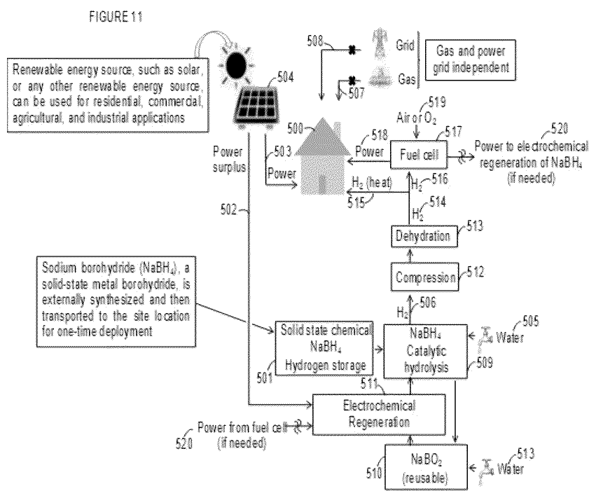

An overall description of the method is shown in FIG. 1. This figure illustrates a flowchart of a method for storage and supply of hydrogen using solid state chemicals, in accordance with some embodiments.

The method may include a step 10 of synthesizing a sodium borohydride (NaBH₄) mass from a natural resource. Further, the natural resource may include borax.

The method may include a step 11 of producing at least one solid state hydrogen carrier using the sodium borohydride mass.

The method may include a step 12 of chemically extracting a hydrogen volume from the at least one solid state hydrogen carrier by reacting the sodium borohydride mass with water in a presence of a catalyst. Further, the chemically extracting may produce a mass of sodium metaborate as a byproduct.

The method may include a step 13 to regenerate the sodium borohydride mass from the sodium metaborate mass using electrochemical means.

The method may include a step 14 of compressing and processing the hydrogen volume in a dehydrator to remove moisture to obtain a usable quantity of hydrogen.

In some embodiments, the usable quantity of hydrogen may be directly used as heat source or supplied to a fuel cell for electricity production.

A heat and power generation solution from green hydrogen for residential, commercial, farm and industrial applications using solid state chemicals.

In some embodiments, the present disclosure describes an industrial plant that may include the at least one solid state hydrogen carrier configured for supplying heat and/or power to the industrial plant.

In some embodiments, the present disclosure describes a power plant that may include the at least one solid state hydrogen carrier configured for generating electric power.

In some embodiments, the present disclosure describes a data center that may include the at least one solid state hydrogen carrier configured for supplying power to the data center

FIG. 2 is a schematic of a system for storage and supply of hydrogen using solid state chemicals, in accordance with some embodiments. Accordingly, the system may include a first reaction unit 50. Further, a mass of sodium borohydride (or NaBH₄) may be synthesized in the first reaction unit 51. The sodium borohydride mass may be synthesized from a natural resource that may include borax and silica. In some embodiments, the synthesizing of the sodium borohydride mass may include the Bayer process. Further, the synthesis of sodium borohydride mass can be represented by the following chemical equation:

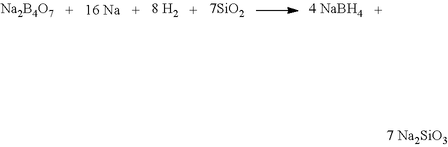

The synthesizing of the sodium borohydride mass may include utilizing a renewable energy resource. Further, at least one solid state hydrogen carrier may be produced using the sodium borohydride mass.

The system may include a second reaction unit 52. In the second reaction unit, a volume of hydrogen 53 may be extracted from the at least one solid state hydrogen carrier by reacting the sodium borohydride mass with water in the presence of a catalyst 54. The chemically extracting may produce a mass of sodium metaborate as a byproduct 57.

The system may include a compression and dehydration units. Further, hydrogen may be compressed 55 and dehydrated in the dehydration unit 56 to obtain a usable quantity of hydrogen.

The system may include a regeneration unit 57. Further, in the regeneration unit, a regenerated sodium borohydride mass may be electrochemically synthesized from the sodium metaborate mass using electrochemical means 58. Some embodiments may involve an electro-synthesis process with a cathode reaction characterized by:

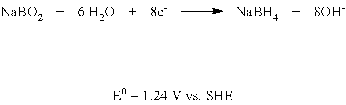

As part of the electro-synthesis process, an anode reaction can occur, which is described as:

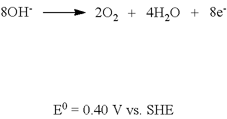

The regenerated sodium borohydride mass may be reacted with water in the second reaction unit 52.

In some embodiments, the usable quantity of hydrogen may be stored in a plurality of transportable packets 59.

In some embodiments, the usable quantity of hydrogen may be supplied to a fuel cell 60 of a hydrogen-powered machine 61. Hydrogen can be used in fuel cells to produce electricity, with the only product being water.

A heat and power generation solution from green hydrogen for residential, commercial, farm and industrial applications using solid state chemicals.

In some embodiments, the present disclosure describes an industrial plant that may include the at least one solid state hydrogen carrier configured for utilizing hydrogen 62 to supply heat 63 and/or power 61 to the industrial plant.

In some embodiments, the present disclosure describes a power plant that may include the at least one solid state hydrogen carrier configured for generating electric power.

In some embodiments, the present disclosure describes a data center that may include the at least one solid state hydrogen carrier configured for supplying power to the data center.

Green hydrogen is generated and stored using only water and renewable energy sources. As a decentralized energy solution, it provides a year-round supply of electricity and heat. Hydrogen storage effectively mitigates daily and seasonal energy fluctuations and supports peak load management. It can also serve as a reliable emergency power source. Furthermore, the process produces only oxygen and water as byproducts, with no CO₂ emissions, ensuring a fully safe method for hydrogen conversion and storage.

Further, the method includes synthesizing sodium borohydride (NaBH₄) from sodium metaborate and water, which makes sodium metaborate regenerable. The energy consumed in the synthesis of the sodium borohydride may be from any source and particularly from a renewable source.

Further, the method includes using the synthesized sodium borohydride as a safe solid medium for storing hydrogen.

Further, the method includes reacting sodium borohydride with water in the presence of a catalyst to yield H₂ at the consumption site. The reaction also produces sodium metaborate as a byproduct, which can be regenerated back into sodium borohydride using electrochemical means. It is preferable to use energy from a renewable source to facilitate this regeneration process. Further, the hydrogen produced from the reaction between sodium borohydride and water will undergo compression and further processing in a dehydrator to remove any remaining moisture. This purified hydrogen can be utilized for heating purposes on-site. Further, the purified hydrogen can also be directly fed to a hydrogen fuel cell to generate electricity to use locally. In addition to its use as a fuel source for hydrogen fuel cells, purified hydrogen can also be employed for stationary power generation, including emergency and backup power services. This can be achieved by utilizing hydrogen as a fuel in power plants, either through direct use or blending it into the fuel stream. Further, in some embodiments, hydrogen can be supplied as fuel to hydrogen fuel cell for distributed power generators in smart grid construction. The reactants for the hydrogen fuel cell are air or oxygen and hydrogen, and the only product is water. Further, the regeneration of sodium metaborate to sodium borohydride involves adding hydrogen back into sodium metaborate.

Further, sodium borohydride (NaBH₄) is synthesized from sodium metaborate and water. The energy consumed in the synthesis of the sodium borohydride is from any source and particularly from a renewable source.

Among chemical hydrides, sodium borohydride (NaBH₄) is a well-known hydrogen source due to its high hydrogen storage capacity of 10.6%. In combination with water, it releases even more hydrogen, making it comparable to a hydrogen carrier medium that can store up to 21.3 wt % of hydrogen:

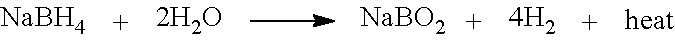

In addition to its hydrogen storage capabilities, NaBH₄ can also be safely and stably dissolved in an alkaline aqueous solution over long periods of time. When necessary, the hydrogen can be released on demand by hydrolyzing NaBH₄ using a heterogeneous catalyst. Cobalt, ruthenium, and nickel are among the catalysts that have been extensively researched. Catalysts based on non-noble metals, especially metal borides (Co—B, Ni—B, etc.) received considerable attention due to their high efficiency, low cost, and simple preparation methods.

The aqueous electrosynthesis of NaBH₄, starting from NaBO₂, is described by the following reaction occurring at the cathode and anode in an electrochemical cell.

### Cathode Reaction:

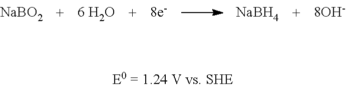

### Anode Reaction:

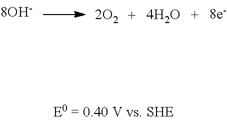

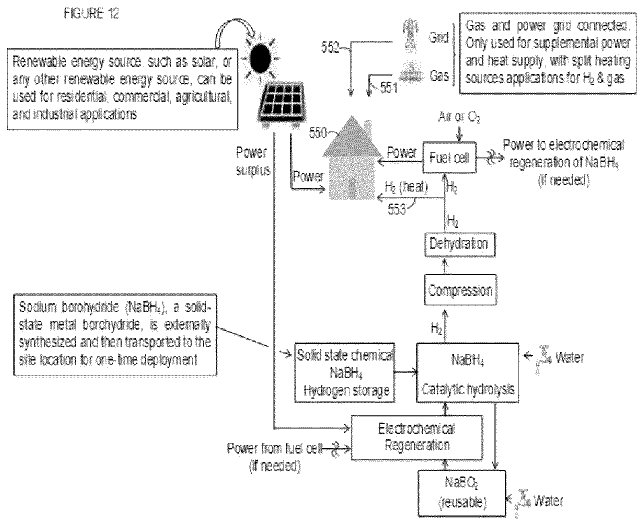

FIG. 3 shows a flowchart of a method and system for distributed generation and storage of green hydrogen, in accordance with some embodiments. A heat and power generation solution from green hydrogen for residential, commercial, farm and industrial applications using solid state chemicals.

Further, the method may include a step 100 of synthesizing a sodium borohydride (NaBH₄) mass 103 from a sodium metaborate and water 101 by electrochemical method. Further, the synthesizing of the sodium borohydride mass may include utilizing energy from a renewable source 102.

Further, the method may include producing at least one solid state hydrogen carrier using the sodium borohydride mass. Further, the at least one solid state hydrogen carrier may be transportable to a consumption site.

Further, the method may include a step 104 of chemically extracting a hydrogen volume from the at least one solid state hydrogen carrier by reacting the sodium borohydride mass with water 105 in a presence of a catalyst. Further, the chemically extracting may produce a mass of sodium metaborate 106 as a byproduct.

Further, the method may include a step 107 to regenerate the sodium borohydride mass from the sodium metaborate mass using electrochemical means. Further, the power from the electrochemical regeneration may come from a renewable source 108.

Further, the method may include a step 109 of compression and further processing the hydrogen volume in a dehydrator 110 to remove moisture to obtain a usable quantity of hydrogen.

Further, in some embodiments, the usable quantity of hydrogen may be stored in a plurality of transportable packets.

Further, the method may include a step to store the usable quantity of hydrogen in a hydrogen storage unit. Furthermore, the hydrogen storage unit may be transportable to a plurality of destinations.

Further, the processed hydrogen can be utilized for heating purposes 111 within the residential setting or nearby premises. Further, the processed hydrogen can be utilized for heating purposes in commercial premises. Further, the processed hydrogen can be utilized for heating purposes in farm/agricultural settings. Further, the processed hydrogen can be utilized for heating purposes in industrial settings. Further, the processed hydrogen can be utilized for heating purposes in hydrogen refueling facilities. Further, the processed hydrogen can be utilized for heating purposes in offshore facilities. In offshore applications, seawater can serve as a resource for synthesizing the sodium borohydride.

Further, the processed hydrogen can be utilized for transportation vehicle refueling purposes in hydrogen refueling facilities.

Further, the processed hydrogen can be directly fed into a hydrogen fuel cell 112 to generate electricity 113 within the residential setting or nearby premises. Further, the electricity generated from the hydrogen fuel cell can be used in the electrochemical regeneration of the sodium borohydride mass from the sodium metaborate mass. Further, the processed hydrogen can be directly fed into a hydrogen fuel cell to generate electricity for commercial premises. Further, the processed hydrogen can be directly fed into a hydrogen fuel cell to generate electricity in farm/agricultural settings. Further, the processed hydrogen can be directly fed into a hydrogen fuel cell to generate electricity in industrial settings. Further, the processed hydrogen can be directly fed into a hydrogen fuel cell to generate electricity in hydrogen refueling facilities. Further, the processed hydrogen can be directly fed into a hydrogen fuel cell to generate electricity for offshore facilities. Further, the processed hydrogen can be directly fed into an ammonia production facility for the synthesis of ammonia. Further, the processed hydrogen can be directly fed into a methanol production facility for the synthesis of methanol. Further, the processed hydrogen can be directly fed into a processing facility for the production of synthetic fuels and chemicals. Further, the hydrogen can be used together with carbon dioxide (CO₂) to produce synthetic fuels and chemicals. Further, the processed hydrogen can be directly fed into a processing facility, such as one utilizing the Fischer-Tropsch reaction, for the production of synthetic fuels, wax and chemicals. Further, the hydrogen can be combined with carbon monoxide (CO), which was initially generated from carbon dioxide (CO₂), to make synthesis gas. This synthesis gas can be utilized in processes such as those employing the Fischer-Tropsch reaction to produce synthetic fuels, wax, and chemicals. In offshore applications, seawater can serve as a resource for synthesizing the sodium borohydride.

Further, in some embodiments, the usable quantity of hydrogen may be supplied to a fuel cell of a hydrogen-powered machine.

Further, in some embodiments, the present disclosure describes an electric vehicle that may include the at least one solid state hydrogen carrier configured for supplying power to the electric vehicle.

Further, in some embodiments, the present disclosure describes an industrial plant that may include the at least one solid state hydrogen carrier configured for supplying heat to the industrial facility.

Further, in some embodiments, the present disclosure describes an industrial plant that may include the at least one solid state hydrogen carrier configured for supplying power using a fuel cell or a hydrogen based power generator to the industrial facility.

Further, in some embodiments, the present disclosure describes a power plant that may include the at least one solid state hydrogen carrier configured for generating electric power.

Further, in some embodiments, the present disclosure describes a data center that may include the at least one solid state hydrogen carrier configured for supplying power using a fuel cell or a hydrogen based power generator to the data center.

Further, in the regeneration unit 107, a regenerated sodium borohydride mass may be electro-synthesized from the sodium metaborate mass using electrochemical means. Further, some embodiments may involve an electro-synthesis process with a cathode reaction characterized by:

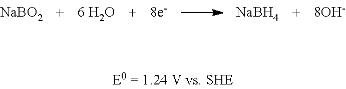

Further, as part of the electro-synthesis process, an anode reaction can occur, which is described as:

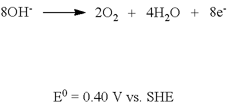

FIG. 4 is a schematic of a distributed or decentralized system for generating and storing green hydrogen, integrating both heat and power supply for use in residential 150, commercial, farm/agricultural and industrial applications using solid state chemicals. It effectively utilizes surplus renewable energy 151 (as the primary power generation 152 is directly used on the premises), such as solar 153, or any other renewable energy source, and available water resources 154 in the reaction unit 155 to produce sodium borohydride 156 and store hydrogen in the form of sodium borohydride 156, a solid state hydrogen storage carrier, in accordance with some embodiments. Further, the system remains independent of the external gas 157 and power grid infrastructure 158. Accordingly, the system may include a first reaction unit 155. Further, a mass of sodium borohydride (NaBH₄) 156 may be synthesized in the first reaction unit. Further, the sodium borohydride 156 mass may be synthesized from sodium metaborate and water electrochemically in reaction unit 155. The sodium metaborate can be transported and positioned onsite for one-time deployment 159 to produce the sodium borohydride. Additionally, sodium borohydride can be synthesized from a naturally occurring resource, such as borax, which can also be transported and placed onsite. Further, the synthesizing of the sodium borohydride mass may include utilizing a renewable energy resource 160. Further, at least one solid state hydrogen carrier may be produced using the sodium borohydride mass. Further, the at least one solid state hydrogen carrier may be transportable to a consumption site.

Further, the system may include a second reaction unit 161. Further, in the second reaction unit 161, a volume of hydrogen may be extracted 162 from the at least one solid state hydrogen carrier by reacting the sodium borohydride mass with water 163 in the presence of a catalyst. Further, the chemically extracting may produce a mass of sodium metaborate 164 as a byproduct.

Further, the method may include a step 165 to regenerate the sodium borohydride mass 161 from the sodium metaborate mass 164 and water 166 using electrochemical means 165. Further, the power 167 for the electrochemical regeneration may come from a renewable source.

Further, the system may include a step 168 of compression and further processing the hydrogen volume in a dehydrator 169 to remove moisture to obtain a usable quantity of hydrogen 170.

Further, the system may include a step to store the usable quantity of hydrogen in a hydrogen storage unit. Furthermore, the hydrogen storage unit may be transportable to a plurality of destinations.

Further, the processed hydrogen 170 can be utilized for heating purposes 171 within the residential 150 setting or nearby premises. Further, the processed hydrogen can be utilized for heating purposes in commercial premises. Further, the processed hydrogen can be utilized for heating purposes in farm/agricultural settings. Further, the processed hydrogen can be utilized for heating purposes in industrial settings. Further, the processed hydrogen can be utilized for heating purposes in hydrogen refueling facilities. Further, the processed hydrogen can be utilized for heating purposes in offshore facilities. In offshore applications, seawater can serve as a resource for synthesizing the sodium borohydride.

Further, the processed hydrogen can be utilized for transportation vehicle refueling purposes in hydrogen refueling facilities.

Further, the processed hydrogen can be directly fed into an ammonia production facility for the synthesis of ammonia. Further, the processed hydrogen can be directly fed into a methanol production facility for the synthesis of methanol. Further, the processed hydrogen can be used together with carbon dioxide (CO₂) and can be directly fed into a processing facility for the production of synthetic fuels and chemicals. Further, the hydrogen can be combined with carbon monoxide (CO), which was initially generated from carbon dioxide (CO₂), to make synthesis gas. Further, this synthesis gas can be utilized in processes such as those employing the Fischer-Tropsch reaction to produce synthetic fuels, wax, and chemicals.

Further, the processed hydrogen 170 can be directly fed 172 into a hydrogen fuel cell 173 to generate electricity 174 for the residential setting 150 or nearby premises. The reactants for the hydrogen fuel cell are air or oxygen 175 and hydrogen 172, and the only product is water. Further, the electricity generated 176 from the hydrogen fuel cell can also be used in the electrochemical regeneration 165 of the sodium borohydride mass 161 from the sodium metaborate mass 164. Further, the processed hydrogen can be directly fed into a hydrogen fuel cell to generate electricity for commercial premises. Further, the processed hydrogen can be directly fed into a hydrogen fuel cell to generate electricity in farm/agricultural settings. Further, the processed hydrogen can be directly fed into a hydrogen fuel cell to generate electricity in industrial settings. Further, the processed hydrogen can be directly fed into a hydrogen fuel cell to generate electricity in hydrogen refueling facilities. Further, the processed hydrogen can be directly fed into a hydrogen fuel cell to generate electricity for offshore facilities. In offshore applications, seawater can serve as a resource for synthesizing the sodium borohydride.

FIG. 5 is a schematic of a distributed or decentralized system for generating and storing green hydrogen, integrating both heat and power supply for use in residential 200, commercial, farm/agricultural and industrial applications using solid state chemicals. This configuration aligns with the description outlined in FIG. 4, with the distinction that is connected with external supply of natural gas 201 and power 202, which are primarily utilized as supplementary sources for power and heat. Further, the system utilizes distinct heating sources for hydrogen 203, which is produced on-site, and gas 201, which is externally supplied.

FIG. 6 is a schematic of a distributed or decentralized system for generating and storing green hydrogen, integrating both heat and power supply for use in residential 250, commercial, farm/agricultural and industrial applications using solid state chemicals. This configuration aligns with the description outlined in FIG. 4, with the distinction that the green hydrogen 251 is blended with externally supplied natural gas 252 using the green hydrogen-gas mixture 253 for heating purposes keeping the system independent of the external power grid infrastructure 254. This system is a low CO₂ generation heating solution. Further, the system involves the integration of green hydrogen 251 with externally sourced natural gas 252, utilizing the resulting green hydrogen-gas blend 253 for heating purposes. Further, the system is independent of the external power grid infrastructure 254. Further, the system serves as a distributed or decentralized low CO₂ green hydrogen heat and power supply system for use in residential 250, commercial, farm/agricultural and industrial applications using solid state chemicals.

FIG. 7 is a schematic of a distributed or decentralized system for generating and storing green hydrogen, integrating both heat and power supply for use in residential 300, commercial, farm/agricultural and industrial applications using solid state chemicals. This configuration aligns with the description outlined in FIG. 4, with the distinction that the green hydrogen 301 is blended with externally supplied natural gas 302 using the green hydrogen-gas mixture 303 for heating purposes. Further, the system is also connected to the external power grid infrastructure 304, enabling the use of supplemental power if necessary. Further, the system involves the integration of green hydrogen 301 with externally sourced natural gas 302, utilizing the resulting green hydrogen-gas blend 303 for heating purposes. Further, the system is also connected to the external power grid infrastructure 304 to draw supplemental power if required.

FIG. 8 is a schematic of a distributed or decentralized system for generating and storing green hydrogen, integrating both heat and power supply for use in residential, commercial, farm/agricultural and industrial applications using solid state chemicals 350. This configuration aligns with the description outlined in FIG. 4, with the distinction that the green hydrogen 351 is blended with on-site produced or externally supplied biogas 352 using the green hydrogen-biogas mixture 353 for heating purposes keeping the system independent of the external power grid infrastructure 354. This system is a low CO₂ generation heating solution. Further, the system involves the integration of green hydrogen 351 with on-site produced or externally supplied biogas 352, utilizing the resulting green hydrogen-biogas blend 353 for heating purposes. Further, the system is independent of the external power grid infrastructure 354. Further, the system serves as decentralized low CO₂ generation green hydrogen heat and power supply system for use in residential 350, commercial, farm/agricultural and industrial applications using solid state chemicals.

FIG. 9 is a schematic of a distributed or decentralized system for generating and storing green hydrogen, integrating both heat and power supply for use in residential 400, commercial, farm/agricultural and industrial applications using solid state chemicals. This configuration aligns with the description outlined in FIG. 4, with the distinction that the green hydrogen 401 is blended with on-site produced or externally supplied biogas 402 using the green hydrogen-biogas mixture 403 for heating purposes. The system is also connected to the external power grid infrastructure 404, enabling the use of supplemental power if necessary. Further, the system involves the integration of green hydrogen 401 with on-site produced or externally supplied biogas 402, utilizing the resulting green hydrogen-biogas blend 403 for heating purposes. Further, the system is connected to the external power grid infrastructure 404 to draw supplemental power if required.

FIG. 10 shows a flowchart of a method and system for distributed generation and storage of green hydrogen using solid state chemicals such as sodium borohydride (NaBH₄), in accordance with some embodiments. The sodium borohydride is externally synthesized 450 and then transported to the site location for one-time deployment. The method and system can also use alternative solid-state chemicals suitable for hydrogen storage, such as any other metal borohydride, in accordance with some embodiments. These metal borohydrides can be also externally synthesized and subsequently transported to the site location for one-time deployment. These metal borohydrides include lithium borohydride (LiBH₄), beryllium borohydride (Be(BH₄)₂) magnesium borohydride (Mg(BH₄)₂), calcium borohydride (Ca(BH₄)₂), zinc borohydride (Zn(BH₄)₂), aluminum borohydride (Al(BH₄)₃), potassium borohydride (KBH₄), scandium borohydride (Sc(BH₄)₃), titanium borohydride (Ti(BH₄)₃), manganese borohydride (Mn(BH₄)₂), zirconium borohydride, (Zr(BH₄)₄), among others.

Further, the method may include a step of chemically extracting 451 a hydrogen volume 452 from the at least one solid state hydrogen carrier by reacting the sodium borohydride mass with water 453 in a presence of a catalyst. Further, the chemically extracting may produce a mass of sodium metaborate as a byproduct 454.

Further, the method may include a step 455 to regenerate the sodium borohydride mass from the sodium metaborate mass using water 456 and electrochemical means. Further, the power for the electrochemical regeneration may come from a renewable source 457.

Further, the method may include a step of compression 458 and further processing the hydrogen volume in a dehydrator 459 to remove moisture to obtain a usable quantity of hydrogen 460.

Further, in some embodiments, the usable quantity of hydrogen may be stored in a plurality of transportable packets.

Further, the method may include a step to store the usable quantity of hydrogen in a hydrogen storage unit. Furthermore, the hydrogen storage unit may be transportable to a plurality of destinations.

Further, the processed hydrogen can be utilized for heating purposes 461 within the residential setting or nearby premises. Further, the processed hydrogen can be utilized for heating purposes in commercial premises. Further, the processed hydrogen can be utilized for heating purposes in farm/agricultural settings. Further, the processed hydrogen can be utilized for heating purposes in industrial settings. Further, the processed hydrogen can be utilized for heating purposes in hydrogen refueling facilities. Further, the processed hydrogen can be utilized for heating purposes in offshore facilities. In offshore applications, seawater can serve as a resource for synthesizing the sodium borohydride.

Further, the processed hydrogen can be utilized for transportation vehicle refueling purposes in hydrogen refueling facilities.

Further, the processed hydrogen can be directly fed into a hydrogen fuel cell 462 to generate electricity 463 for the residential setting or nearby premises. The reactants for the hydrogen fuel cell are air or oxygen 464 and hydrogen 460, and the only product is water. Further, the electricity generated from the hydrogen fuel cell can be used in the electrochemical regeneration of the sodium borohydride mass from the sodium metaborate mass. Further, the processed hydrogen can be directly fed into a hydrogen fuel cell to generate electricity for commercial premises. Further, the processed hydrogen can be directly fed into a hydrogen fuel cell to generate electricity in farm/agricultural settings. Further, the processed hydrogen can be directly fed into a hydrogen fuel cell to generate electricity in industrial settings. Further, the processed hydrogen can be directly fed into a hydrogen fuel cell to generate electricity in hydrogen refueling facilities. Further, the processed hydrogen can be directly fed into a hydrogen fuel cell to generate electricity for offshore facilities. Further, the processed hydrogen can be directly fed into an ammonia production facility for the synthesis of ammonia. Further, the processed hydrogen can be directly fed into a methanol production facility for the synthesis of methanol. Further, the processed hydrogen can be directly fed into a processing facility for the production of synthetic fuels and chemicals. Further, the hydrogen can be used together with carbon dioxide (CO₂) to produce synthetic fuels and chemicals. Further, the processed hydrogen can be directly fed into a processing facility, such as one utilizing the Fischer-Tropsch reaction, for the production of synthetic fuels, wax and chemicals. Further, the hydrogen can be combined with carbon monoxide (CO), which was initially generated from carbon dioxide (CO₂), to make synthesis gas. This synthesis gas can be utilized in processes such as those employing the Fischer-Tropsch reaction to produce synthetic fuels, wax, and chemicals. In offshore applications, seawater can serve as a resource for synthesizing the sodium borohydride.

Further, in some embodiments, the usable quantity of hydrogen may be supplied to a fuel cell of a hydrogen-powered machine.

Further, in some embodiments, the present disclosure describes an electric vehicle that may include the at least one solid state hydrogen carrier configured for supplying power to the electric vehicle.

Further, in some embodiments, the present disclosure describes an industrial plant that may include the at least one solid state hydrogen carrier configured for supplying heat to the industrial facility.

Further, in some embodiments, the present disclosure describes an industrial plant that may include the at least one solid state hydrogen carrier configured for supplying power using a fuel cell or a hydrogen based power generator to the industrial facility.

Further, in some embodiments, the present disclosure describes a power plant that may include the at least one solid state hydrogen carrier configured for generating electric power.

Further, in some embodiments, the present disclosure describes a data center that may include the at least one solid state hydrogen carrier configured for supplying power using a fuel cell or a hydrogen based power generator to the data center.

Further, in the regeneration unit, a regenerated sodium borohydride mass may be electro-synthesized from the sodium metaborate mass using electrochemical means. Further, some embodiments may involve an electro-synthesis process with a cathode reaction characterized by:

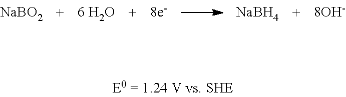

Further, as part of the electro-synthesis process, an anode reaction can occur, which is described as:

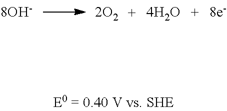

FIG. 11 is a schematic of a distributed or decentralized system for generating and storing green hydrogen, integrating both heat and power supply for use in residential 500, commercial, farm/agricultural and industrial applications using solid state chemicals such as sodium borohydride (NaBH₄). The sodium borohydride is externally synthesized 501 and then transported to the site location for one-time deployment. The method and system can also use alternative solid-state chemicals suitable for hydrogen storage, such as any other metal borohydride, in accordance with some embodiments. It effectively utilizes surplus renewable energy 502 (as the primary power generation 503 is directly used on the premises), such as solar 504, or any other renewable energy source, and available water resources 505 to generate hydrogen 506 from sodium borohydride 501, a solid state hydrogen storage carrier, in accordance with some embodiments. Further, the system remains independent of the external gas 507 and power grid infrastructure 508. Accordingly, the system may include a reaction unit 509. Further, in the reaction unit, a volume of hydrogen 506 may be extracted from the at least one solid state hydrogen carrier by reacting the sodium borohydride mass with water in the presence of a catalyst 509. Further, the chemically extracting may produce a mass of sodium metaborate 510 as a byproduct.

Further, the method may include a step 511 to regenerate the sodium borohydride mass 509 from the sodium metaborate mass 510 using electrochemical means 511. Further, the power 502 for the electrochemical regeneration may come from a surplus renewable energy source.

Further, the system may include a step 512 of compression and further processing the hydrogen volume in a dehydrator 513 to remove moisture to obtain a usable quantity of hydrogen 514.

Further, the system may include a step to store the usable quantity of hydrogen in a hydrogen storage unit. Furthermore, the hydrogen storage unit may be transportable to a plurality of destinations.

Further, the processed hydrogen 514 can be utilized for heating purposes 515 within the residential 500 setting or nearby premises. Further, the processed hydrogen can be utilized for heating purposes in commercial premises. Further, the processed hydrogen can be utilized for heating purposes in farm/agricultural settings. Further, the processed hydrogen can be utilized for heating purposes in industrial settings. Further, the processed hydrogen can be utilized for heating purposes in hydrogen refueling facilities. Further, the processed hydrogen can be utilized for heating purposes in offshore facilities.

Further, the processed hydrogen can be utilized for transportation vehicle refueling purposes in hydrogen refueling facilities.

Further, the processed hydrogen can be directly fed into an ammonia production facility for the synthesis of ammonia. Further, the processed hydrogen can be directly fed into a methanol production facility for the synthesis of methanol. Further, the hydrogen can be used together with carbon dioxide (CO₂) to produce synthetic fuels and chemicals. Further, the hydrogen can be combined with carbon monoxide (CO), which was initially generated from carbon dioxide (CO₂), to make synthesis gas. Further, this synthesis gas can be utilized in processes such as those employing the Fischer-Tropsch reaction to produce synthetic fuels, wax, and chemicals.

Further, the processed hydrogen 514 can be directly fed 516 into a hydrogen fuel cell 517 to generate electricity 518 for the residential setting 500 or nearby premises. The reactants for the hydrogen fuel cell are air or oxygen 519 and hydrogen 516, and the only product is water. Further, the electricity generated 520 from the hydrogen fuel cell can be used in the electrochemical regeneration of the sodium borohydride mass 509 from the sodium metaborate mass 510. Further, the processed hydrogen can be directly fed into a hydrogen fuel cell to generate electricity for commercial premises. Further, the processed hydrogen can be directly fed into a hydrogen fuel cell to generate electricity in farm/agricultural settings. Further, the processed hydrogen can be directly fed into a hydrogen fuel cell to generate electricity in industrial settings. Further, the processed hydrogen can be directly fed into a hydrogen fuel cell to generate electricity in hydrogen refueling facilities. Further, the processed hydrogen can be directly fed into a hydrogen fuel cell to generate electricity for offshore facilities.

FIG. 12 is a schematic of a distributed or decentralized system for generating and storing green hydrogen, integrating both heat and power supply for use in residential 550, commercial, farm/agricultural and industrial applications using solid state chemicals. This configuration aligns with the description outlined in FIG. 11, with the distinction that is connected with external supply of natural gas 551 and power 552, which are primarily utilized as supplementary sources for power and heat. Further, the system utilizes distinct heating sources for hydrogen 553, which is produced on-site, and gas 551, which is externally supplied.

FIG. 13 is a schematic of a distributed or decentralized system for generating and storing green hydrogen, integrating both heat and power supply for use in residential 600, commercial, farm/agricultural and industrial applications using solid state chemicals. This configuration aligns with the description outlined in FIG. 11, with the distinction that the green hydrogen 601 is blended with externally supplied natural gas 602 using the green hydrogen-gas mixture 603 for heating purposes keeping the system independent of the external power grid infrastructure 604. This system is a low CO₂ generation heating solution. Further, the system involves the integration of green hydrogen 601 with externally sourced natural gas 602, utilizing the resulting green hydrogen-gas blend 603 for heating purposes. Further, the system is independent of the external power grid infrastructure 604. Further, the system serves as a distributed or decentralized low CO₂ green hydrogen heat and power supply system for use in residential 600, commercial, farm/agricultural and industrial applications using solid state chemicals.

FIG. 14 is a schematic of a distributed or decentralized system for generating and storing green hydrogen, integrating both heat and power supply for use in residential 650, commercial, farm/agricultural and industrial applications using solid state chemicals. This configuration aligns with the description outlined in FIG. 11, with the distinction that the green hydrogen 651 is blended with externally supplied natural gas 652 using the green hydrogen-gas mixture 653 for heating purposes. Further, the system is also connected to the external power grid infrastructure 654, enabling the use of supplemental power if necessary. Further, the system involves the integration of green hydrogen 651 with externally sourced natural gas 652, utilizing the resulting green hydrogen-gas blend 653 for heating purposes. Further, the system is also connected to the external power grid infrastructure 654 to draw supplemental power if required.

FIG. 15 is a schematic of a distributed or decentralized system for generating and storing green hydrogen, integrating both heat and power supply for use in residential 700, commercial, farm/agricultural, and industrial applications using solid state chemicals. This configuration aligns with the description outlined in FIG. 11, with the distinction that the green hydrogen 701 is blended with on-site produced or externally supplied biogas 702 using the green hydrogen-biogas mixture 703 for heating purposes keeping the system independent of the external power grid infrastructure 704. This system is a low CO₂ generation heating solution. Further, the system involves the integration of green hydrogen 701 with on-site produced or externally supplied biogas 702, utilizing the resulting green hydrogen-biogas blend 703 for heating purposes. Further, the system is independent of the external power grid infrastructure 704. Further, the system serves as decentralized low CO₂ green hydrogen heat and power supply system for use in residential 700, commercial, farm/agricultural and industrial applications using solid state chemicals.

FIG. 16 shows a schematic of a decentralized system for generating and storing green hydrogen, integrating both heat and power supply for use in residential 750, commercial, farm/agricultural and industrial applications using solid state chemicals. This configuration aligns with the description outlined in FIG. 11, with the distinction that the green hydrogen 751 is blended with on-site produced or externally supplied biogas 752 using the green hydrogen-biogas mixture 753 for heating purposes. The system is also connected to the external power grid infrastructure 754, enabling the use of supplemental power if necessary. Further, the system involves the integration of green hydrogen 751 with on-site produced or externally supplied biogas 752, utilizing the resulting green hydrogen-biogas blend 753 for heating purposes. Further, the system is connected to the external power grid infrastructure 754 to draw supplemental power if required.

Although the invention has been explained in relation to its preferred embodiment, it is to be understood that many other possible modifications and variations can be made without departing from the spirit and scope of the invention.

What is claimed is:

1. A method for storage and supply of hydrogen using solid-state chemicals, comprising:
a. Synthesizing a sodium borohydride (NaBH₄) mass from a natural resource or from sodium metaborate (NaBO₂) and water through an electrochemical method;
b. Producing at least one solid-state hydrogen carrier using the sodium borohydride mass;
c. Transporting the at least one solid-state hydrogen carrier to a hydrogen utilization site;
d. Chemically extracting a hydrogen volume from the at least one solid-state hydrogen carrier by reacting the sodium borohydride mass with water in the presence of a catalyst, thereby producing sodium metaborate as a byproduct;
e. Processing the hydrogen volume by compressing it and removing any remaining moisture to obtain a usable quantity of hydrogen;
f. Storing the usable quantity of hydrogen in a hydrogen storage unit that may be transportable to a plurality of destinations or in transportable packets; and
g. Regenerating the sodium borohydride mass from the sodium metaborate mass using electrochemical means.

2. The method of claim 1, wherein the synthesized sodium borohydride is produced from natural resources, such as borax.

3. The method of claim 1, wherein the synthesis of sodium borohydride mass utilizes a renewable energy resource.

4. The method of claim 1, wherein the usable quantity of hydrogen is supplied to a hydrogen fuel cell for use in hydrogen-powered machines, vehicles, ships, submarines, planes, or spacecraft.

5. The method of claim 1, further comprising supplying power to electric vehicles, industrial plants, power plants, or data centers using at least one solid-state hydrogen carrier.

6. The method of claim 1, wherein hydrogen gas is further stored in corresponding tanks using methods such as compression, liquefaction, or adsorption onto a solid porous adsorbent.

7. A method for distributed storage and supply of hydrogen gas as a renewable fuel source, comprising:
a. Synthesizing sodium borohydride (NaBH₄) externally and transporting it to the site location for hydrogen production;
b. Storing the sodium borohydride at the site location for short-term or long-term periods;
c. Extracting hydrogen gas from the stored sodium borohydride at the utilization site by reacting it with water in the presence of a catalyst, producing sodium metaborate as a byproduct;
d. Processing the hydrogen gas by compressing it and removing any remaining moisture prior to its utilization; and
e. Regenerating sodium borohydride from sodium metaborate using electrochemical means powered by renewable energy.

8. The method of claim 7, wherein the hydrogen gas is utilized for generating electricity and heat in residential, commercial, farm/agricultural, or industrial settings.

9. The method of claim 7, wherein the hydrogen gas is fed into a fuel cell to generate electricity for local use or to supply power to the grid during peak demand periods.

10. The method of claim 7, wherein the hydrogen gas is used in hydrogen refueling facilities.

11. The method of claim 7, wherein the processed hydrogen is blended with biogas to form a mixed fuel or used directly in ammonia production, methanol production, or synthetic fuel synthesis.

12. The method of claim 7, wherein the hydrogen gas is used with carbon monoxide (CO) or carbon dioxide (CO₂) to produce synthesis gas or other synthetic fuels.

13. A method for distributed or decentralized storage and supply of hydrogen gas as a renewable fuel source, comprising:
a. Producing a solid chemical, such as sodium borohydride or another metal borohydride, that effectively stores hydrogen using excess renewable energy;
b. Storing the solid chemical for future utilization, with a storage period ranging from short-term to long-term;
c. Extracting hydrogen gas from the stored solid chemical at a utilization site by reacting the sodium borohydride mass with water in the presence of a catalyst, producing sodium metaborate as a byproduct;
d. Processing the hydrogen gas by compressing it and removing any remaining moisture prior to its utilization; and
e. Regenerating sodium borohydride from sodium metaborate using electrochemical means, preferably utilizing renewable energy for the regeneration process.

14. The method of claim 13, wherein the hydrogen gas is blended with biogas to form a mixed fuel for heating purposes.

15. The method of claim 13, wherein the processed hydrogen is utilized for heating purposes in offshore facilities or fed into hydrogen fuel cells to generate electricity for offshore facilities.

16. The method of claim 13, wherein the processed hydrogen is fed directly into an ammonia production facility for the synthesis of ammonia.

17. The method of claim 13, wherein the processed hydrogen is fed directly into a methanol production facility for the synthesis of methanol.

18. The method of claim 13, wherein the processed hydrogen is fed directly into a processing facility for the production of synthetic fuels and chemicals.

19. The method of claim 13, wherein the processed hydrogen is used with carbon dioxide (CO₂) to produce synthetic fuels and chemicals.

20. The method of claim 13, wherein the processed hydrogen is combined with carbon monoxide (CO) generated from carbon dioxide (CO₂) to produce synthesis gas.
